(19) United States
(12) Patent Application Publication
(10) Pub. No.: US 2020/0354791 A1
(43) Pub. Date: Nov. 12, 2020

(54) METHODS FOR PROGNOSIS OR TREATMENT OF PARKINSON'S DISEASE

(71) Applicant: BIO SHAI LTD., Yokneam Elite (IL)

(72) Inventors: Jennifer YARDEN, Moshav Maor (IL); Nir DOTAN, Shoham (IL); Danit MECHLOVICH, Binyamina (IL); Jose Martin RABEY, Tel Aviv (IL); Moussa B. H. YOUDIM, Nesher (IL)

(73) Assignee: BIO SHAI LTD., Yokneam Elite (IL)

(21) Appl. No.: 16/762,163

(22) PCT Filed: Nov. 8, 2018
(86) PCT No.: PCT/IL2018/051205

§ 371 Date: May 7, 2020

Related U.S. Application Data
(60) Provisional application No. 62/583,132, filed on Nov. 8, 2017.

(51) Int. Cl. C12Q 1/6883

(52) U.S. Cl. CPC C12Q 1/6883; A61K 31/166; C12Q 2600/158; C12Q 2600/118

(57) ABSTRACT

Provided herein are methods for prognosing and treating a patient with Parkinson's disease (PD) or Parkinsonian symptoms. The prognosis and appropriate treatments can be determined by correlating the level of gene expression of SKP1a, UBE2K, ALDH1A1, PSMC4, HSPA8 and LAMB2 with a Parkinson's Disease rating scale, thereby prognosing slow or rapid progression of the symptoms or disease.

### CROSS-REFERENCE TO RELATED APPLICATIONS

This application claims the benefit of U.S. Provisional Patent Application No. 62/583,132 filed on Nov. 8, 2017; the contents of which are incorporated by reference herein in their entirety.

### FIELD

Provided herein are methods for prognosing and treating a patient with Parkinson's disease (PD) or Parkinsonian symptoms.

### BACKGROUND

Parkinson's Disease (PD) is a disorder of the central nervous system with a relatively high prevalence in adults aged over 60. PD symptoms include movement disorders such as tremor and rigidity. The etiology of PD is heterogeneous, genetic, and multi-factorial, resulting in a highly variable clinical course, spanning from a slowly progressive, benign course to a rapidly progressive, disabling disease. (Lawton 2015).

PD is a difficult disease to accurately diagnose and can be confused with many disorders. Usually, at early stages, PD is most difficult to diagnose but diagnostic accuracy improves as clinical symptoms develop (Masano 2012).

There are a variety of treatment options available for PD patients. Of the medications available for treatment of PD, there are a number of classes which have been shown to relieve the disease's symptoms, including Carbidopa/Levodopa, Dopamine agonists, anticholinergics, MAO-B inhibitors, COMT inhibitors and other medications (Parkinson's Disease Foundation Overview, 2015). A PD patient may be treated, simultaneously or concomitantly with multiple classes and/or multiple medications at various times of the day or various stages of the disease. In addition to medications, surgical intervention for PD patients, such as Deep Brain Stimulation, is also available.

Although symptomatic therapy can provide benefit for many years, PD is a progressive disorder that will eventually result in significant morbidity. Knowledge of the features that predict the rate of progression may empower clinicians to better counsel patients regarding prognosis, treatment and life expectancy. Improvement in prognosis accuracy and the ability to predict the rate of progression would impact on the ability to choose the most beneficial treatment with the least number of unnecessary side-effects. Thus, a continuing need exists for early prognosis of PD progression rates and the associated determination and implementation of appropriate treatments.

### SUMMARY

Described herein are methods for prognosing and treatment of a patient presenting with early stage Parkinson's Disease (PD) symptoms. The disclosed method for prognosing a patient presenting with early stage Parkinson's Disease (PD) symptoms, includes: determining a level of expression of at least one gene selected from the group consisting of SKP1a, UBE2K, ALDH1A1, PSMC4, HSPA8 and LAMB2 in a biological sample from a patient, and correlating the level of gene expression with a Parkinson's Disease rating scale, thereby prognosing the patient with slow or rapid progression of the symptoms or disease.

Further methods provided herein relate to a method of reducing Parkinson's disease symptoms in a patient presenting with early stage Parkinson's Disease symptoms which includes: determining a level of expression of at least one gene selected from the group consisting of SKP1a, UBE2K, ALDH1A1, PSMC4, HSPA8 and LAMB2 in a biological sample from a patient; from the level of expression of the at least one gene determining whether patient's disease or symptoms are predicted to progress rapidly or slowly; and administering to the patient a therapeutic effective amount of a symptom reducing medication appropriate for the slow or rapid progression of the symptom, thereby reducing Parkinson's disease symptoms in a patient with slow or rapid progression of the symptoms or disease.

Methods provided herein further relate a method for prognosing a patient presenting with early stage Parkinson's Disease symptoms, which includes: determining a level of expression of at least one gene selected from the group consisting of ALDH1A1, LAMB2, SKP1a, and UBE2K in a biological sample from a patient; and correlating the level of gene expression with a score from a rating scale selected from the group consisting of: Hoehn and Yahr scale (H&Y), Modified Schwab and England Activities of Daily Living (Modified Schwab and England), and Unified Parkinson Disease Rating Scale (UPDRS), thereby prognosing the patient with slow or rapid progression of the disease.

Further methods provided herein relate to a method for prognosing a patient presenting with Parkinson's Disease-related cognitive decline, including: determining a level of expression of at least one gene selected from the group consisting of HSPA8 and SKP1a in a biological sample from a patient; and correlating the level of gene expression with a score from a rating scale selected from the group consisting of: Montreal Cognitive Assessment (MoCA), Mini Mental State Examination (MMSE), Hopkins Verbal Learning Test (HVLT) and University of Pennsylvania Smell Identification Test (UPSIT), thereby prognosing the patient with PD-related cognitive decline.

Additional methods described herein relate to a method for prognosing a patient presenting with Parkinson's Disease-related symptoms, including: determining a level of expression of at least one gene selected from the group consisting of LAMB2 and SKP1a in a biological sample from a patient; and correlating the patient's level of gene expression with unfavorable results of MDS UPDRS, thereby prognosing the patient with rapid progression of the PD-related Dyskinesia.

The foregoing and other objects, features, and advantages will become more apparent from the following detailed description, which proceeds with reference to the accompanying figures.

### BRIEF DESCRIPTION OF THE DRAWINGS

FIG. 1 depicts a flow diagram showing a method of prognosis for a patient having early stage Parkinson's disease.

FIG. 2 is a scatter plot depicting the patient's blood analyses for genes ALDH1a, and PSMC4. The analyses depicts scatter plots showing the significant correlation of 3 years post-baseline Modified Schwab and England score with baseline values (expressed in delta-delta CT, ddCT) of ALDH1a in FIG. 2a (P-Value=0.0012, rho=−0.208, 95% CI −0.326 to −0.0839); and PSMC4 in FIG. 2b (P=0.006, rho=−0.178, 95% CI −0.298 to −0.0522).

FIG. 3A shows a receiver operator characteristics (ROC) curve of CL-Prediction baseline values and age for detecting PD patients who reached Modified Schwab and England ≤70% at 3 years post-baseline. Cutoffs were determined at the Youden index (specificity=81.7, sensitivity=80.0) and at high specificity (specificity=93.3, sensitivity=46.7). Area under the Curve (AUC) for CL-Prediction was 0.852 (95% CI 0.800-0.894, P-Value<0.0001) and for age was 0.731 (95% CI, 0.670-0.786, P-Value=0.0005).

FIGS. 3B-3D depict Kaplan-Meir curves of CL-Prediction for predicting PD patients who have faster time, in days to ≤70% Modified Schwab and England. Using the Youden index cutoff, as shown in FIG. 3B, the hazard ratio (HR+) of reaching the endpoint was 13.57 (95% CI, 4.06-45.38), negative hazard ratio (HR−) was 0.074 (95% CI, 0.022-0.25). FIG. 3C shows using the high specificity cutoff, the HR+ of reaching the endpoint was 7.42 (95% CI, 1.43-38.5), HR− was 0.13 (95% CI, 0.026-0.70). FIG. 3D shows using both the both the Youden Index (Intermediate-Pos) and high specificity (High-Pos) cutoffs, the HR+ at the High-Pos cutoff was 17.08 (95% CI, 3.24-89.89), the HR+ at the Intermediate-Pos cutoff was 10.55 (95% CI, 2.22-50.12) and HR− was 0.059 (95% CI, 0.11-0.31).

FIG. 4 depicts a Kaplan-Meir curve of CL-Prediction for predicting PD patients who have faster disease progression as shown by time to increase of Unified Parkinson's disease rating scale (UPDRS) score by 17 points.

FIG. 5 is the qPCR results of a gene analyses for HSPA8, PSMC4 and SKP1a. The box plots represent the ddCT levels at Visit 4 (1 year post-baseline) of PD patients by MoCA below 24 vs. equal and above 24.

FIG. 6 shows a receiver operator characteristics (ROC) curve relating to the cognitive predictive classifier associated with patients who progressed to <24 MoCA within three years of diagnosis by 3-years post baseline, as compared to ROC curves associated with individual gene expression levels alone, age of patient, and UPSIT scores.

FIG. 7 depicts a Kaplan-Meir curve of the cognitive predictive classifier depicting difference in days until endpoint (<24 MoCA score) in patients who were positive or negative for the cognitive predictive classifier.

FIGS. 8A-8D show the analyses of qPCR results for genes LAMB2 (FIG. 8A) and of SKP1a (FIG. 8C) showing correlations between expression levels of the specified genes and development of dyskinesia; and ROC curves relating to prediction of Dyskinesia based on expression levels at baseline for LAMB2 (FIG. 8B) and of SKP1a (FIG. 8D).

FIGS. 9A-9B show the CL-Prediction PP baseline values of PD patients who reached H&Y stage≥3. FIG. 9A depicts PD-prediction baseline levels in Box plot compared to H&Y stage at 3 years-post shows CL-Prediction PP baseline values were significantly higher in baseline blood of PD patients who reached H&Y stage≥3 than PD patients whose H&Y stage<at 3 years post-baseline. FIG. 9B depicts a Kaplan-Meier curve for predicting time to H&Y stage≥3 using the Youden Index Cutoff. CL-Prediction significantly distinguished patients with faster time to H&Y stage≥3 using Cutoff-1 with an HR+ of 4. and HR− of 0.24.

### DETAILED DESCRIPTION

### I. Terms

Unless otherwise noted, technical terms are used according to conventional usage, which for example can be found in Benjamin Lewin, Genes V, published by Oxford University Press, 1994 (ISBN 0-19-854287-9); Kendrew et al. (eds.). The singular terms “a”, “an”, and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.” In case of conflict, the present specification, including explanations of terms, will control. In addition, all the materials, methods, and examples are illustrative and not intended to be limiting.

Administration: The introduction of a composition into a subject by a chosen route. Administration of an active compound or composition can be by any route known to one of skill in the art. Administration can be local or systemic. Examples of local administration include, but are not limited to, topical administration, subcutaneous administration, intramuscular administration, intrathecal administration, intrapericardial administration, intra-ocular administration, topical ophthalmic administration, or administration to the nasal mucosa or lungs by inhalational administration. In addition, local administration includes routes of administration typically used for systemic administration, for example by directing intravascular administration to the arterial supply for a particular organ. Thus, in particular embodiments, local administration includes intra-arterial administration and intravenous administration when such administration is targeted to the vasculature supplying a particular organ. Local administration also includes the incorporation of active compounds and agents into implantable devices or constructs, such as vascular stents or other reservoirs, which release the active agents and compounds over extended time intervals for sustained treatment effects.

Systemic administration includes any route of administration designed to distribute an active compound or composition widely throughout the body via the circulatory system. Thus, systemic administration includes, but is not limited to intra-arterial and intravenous administration. Systemic administration also includes, but is not limited to, topical administration, subcutaneous administration, intramuscular administration, or administration by inhalation, when such administration is directed at absorption and distribution throughout the body by the circulatory system.

Biological Sample: Any sample that may be obtained directly or indirectly from an organism, including whole blood, plasma, serum, tears, mucus, saliva, urine, pleural fluid, spinal fluid, gastric fluid, sweat, semen, vaginal secretion, sputum, fluid from ulcers and/or other surface eruptions, blisters, abscesses, tissues, cells (such as, fibroblasts, peripheral blood mononuclear cells, or muscle cells), organs, and/or extracts of tissues, cells (such as, fibroblasts, peripheral blood mononuclear cells, or muscle cells), bone marrow, or organs. A sample is collected or obtained using methods well known to those skilled in the art.

Cognitive Decline: Decrease in cognition, which may be associated with Parkinson's Disease. Cognitive decline may be evident in decline in short term and working memory, visuospatial abilities, executive function, attention, concentration, language and orientation, or in combinations thereof.

Control: A reference standard. A control can be a known value indicative of basal expression of a diagnostic molecule such as gene described, sometimes referred to as a “predetermined value”. In particular examples a control sample is taken from a subject that is known not to have a disease or condition. In other examples a control is taken from the subject being diagnosed, but at an earlier time point, either before disease onset or prior to or at an earlier time point in disease treatment. A difference between a test sample and a control can be an increase or conversely a decrease. The difference can be a qualitative difference or a quantitative difference, for example a statistically significant difference. In some examples, a difference is an increase or decrease, relative to a control, of at least about 10%, such as at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Diagnosis: The process of identifying a disease or a predisposition to developing a disease or condition, for example the methods disclosed herein. The conclusion reached through that process is also called “a diagnosis.” A subject diagnosed with a disease or condition is understood to be “afflicted” with the disease or condition.

Disease Progression: Parkinson's Disease is a progressive disease which worsens with time. The progression could be defined as rapid or slow. Disease progression may be evident by comparing a symptom (or combination of symptoms) of PD in a patient at a point in time, and comparing the symptom(s) severity at a later point in time. The etiology of PD is heterogeneous, genetic, and multi-factorial, resulting in a highly variable clinical course, spanning from a slowly progressive, benign course to a rapidly progressive, disabling disease.

Dyskinesia: Abnormal, uncontrolled, or involuntary movement. It can affect one body part, such as an arm, leg or the head, or it can spread over the entire body. Dyskinesia can appear as fidgeting, writhing, wriggling, head bobbing or body swaying. It can occur to different degrees of severity.

Early Stage Parkinson's disease: Parkinson's Disease within three years of diagnosis of disease in the patient.

Marker: A molecule present in a biological sample of a patient. The marker's quantity in the biological sample may be analyzed and compared to a threshold level, or to a known level. Increased or decreased level of the marker in the biological sample relative to a known level may be indicative of presence of or tendency to develop a disease.

Patient: A patient capable of, prone to, or predisposed to developing a disease or condition. It is understood that a patient already having or showing symptoms of a disease or condition is considered “susceptible” since they have already developed it.

Prognosis: A probable outcome or course of disease, or the process for determining a probable outcome or course of disease. In particular embodiments, prognosis is the outcome or course of the given disease in the absence of treatment; in other embodiments, it is the outcome course of the disease following a particular treatment.

Therapeutically effective amount: A quantity of compound sufficient to achieve a desired effect in a subject being treated. An effective amount of a compound may be administered in a single dose, or in several doses, for example daily, during a course of treatment. However, the effective amount will be dependent on the compound applied, the subject being treated, the severity and type of the affliction, and the manner of administration of the compound. For example, a therapeutically effective amount of an active ingredient can be measured as the concentration (moles per liter or molar-M) of the active ingredient (such as a small molecule, peptide, protein, or antibody) in blood (in vivo) or a buffer (in vitro) that produces an effect.

### II. Overview of Several Embodiments

Described herein are methods for prognosing a patient presenting with early stage Parkinson's Disease (PD) symptoms, which includes: determining a level of expression of at least one gene such as SKP1a, UBE2K, ALDH1A1, PSMC4, HSPA8 and LAMB2 in a biological sample from a patient, and correlating the level of gene expression with a Parkinson's Disease rating scale, thereby prognosing the patient with slow or rapid progression of the symptoms or disease.

In some embodiments, the symptoms can be resting tremor, bradykinesia, cognitive decline, rigidity, asymmetric resting tremor and asymmetric bradykinesia.

In some embodiments, the Parkinson's Disease rating scale can be Hoehn and Yahr scale (H&Y), Modified Schwab and England Activities of Daily Living (Modified Schwab and England), and Unified Parkinson Disease Rating Scale (UPDRS).

In particular embodiments, rapid progression correlates with a lower Modified Schwab and England Scale and/or increased H&Y score correlates with decreased baseline expression of the gene selected from the group consisting of ALDH1A1, LAMB2, SKP1a, and UBE2K prognoses the patient as having rapid progression of the symptoms or disease.

In some embodiments the correlation is determined by a CL-Prediction algorithm comprising: (1.5479*ΔΔCT ALDH1a)+(−0.91861*ΔΔCT LAMB2)+(−0.21651*ΔΔCT UBE2K)+(1.15002*ΔΔCT SKP1a)+(0.11518*Age)−12.4435=predictive probability (PP) value, indicating rapid or slow prognosis of the disease.

In some embodiments a positive PP value indicates a rapid progression of the disease and negative PP value indicates slow progression of the disease.

In particular embodiments, the disease progression is determined by measurement of cognitive decline. In additional embodiments, the degree of cognitive decline can be measured by Montreal Cognitive Assessment (MoCA), Mini Mental State Examination (MMSE), Hopkins Verbal Learning Test (HVLT) and University of Pennsylvania Smell Identification Test (UPS T).

In some embodiments the cognitive decline correlates with increased expression of the genes such as HSPA8 and SKP1a.

In particular embodiments the cognitive decline correlation is determined by the algorithm comprising: (−2.39361*ΔΔCT HSPA8)+(−1.27186*ΔΔCT SKP1a)+(0.15828*Age)+(−0.11884*UPSIT)+1.54744=PP value, indicating rapid or slow cognitive decline. In further embodiments, the cognitive decline correlation is determined by the algorithm comprising: (−2.36482*ΔΔCT HSPA8)+(−1.39981*ΔΔCT SKP1a)+(−0.18071*Age)−1.8556=PP value, indicating rapid or slow cognitive decline

In some embodiments the positive PP value indicates a rapid cognitive decline and negative PP value indicates slow, or minimal cognitive decline.

In some embodiments the disease progression includes development of Dyskinesia, which is measured by the Movement Disorder Society Unified Parkinson Disease Rating Scale (MDS UPDRS). In particular embodiments, the MDS UPDRS correlates with decreased expression of genes LAMB2 and SKP1a.

Further described herein is a method of reducing Parkinson's disease symptoms in a patient presenting with early stage Parkinson's Disease symptoms which includes: determining a level of expression of at least one gene such as SKP1a, UBE2K, ALDH1A1, PSMC4, HSPA8 and LAMB2 in a biological sample from a patient; the gene expression level is used to determine whether patient's disease or symptoms are predicted to progress rapidly or slowly; and administering to the patient a therapeutic effective amount of a symptom reducing medication appropriate for the slow or rapid progression of the symptom, thereby reducing Parkinson's disease symptoms in a patient with slow or rapid progression of the symptoms or disease in a manner most appropriate for the disease prognosis.

In particular embodiments the rapid progression of Parkinson's Disease symptoms is treated with levodopa.

In some embodiments, the rapid progression is associated with dyskinesia and treated with symptom reducing medication such as low doses of levodopa, extended release of levodopa, continuous release of levodopa, and amantadine.

In some embodiments, the rapid progression is associated with cognitive decline and treated with an atypical antipsychotic, a cholinesterase inhibitor, or a glutamine inhibitor.

In some embodiments, slow progression of the disease is not treated with symptom reducing medication such as levodopa, or carbidopa-levodopa. In particular embodiments, slow progression of the disease is treated with symptom reducing medication comprising: dopamine agonists and/or MAO-B inhibitors.

In particular embodiments, treatment also includes surgical therapies such as deep brain stimulation or intentional formation of lesions.

Further described herein is a method for prognosing a patient presenting with early stage Parkinson's Disease symptoms, which includes: determining a level of expression of at least one gene such as ALDH1A1, LAMB2, SKP1a, and UBE2K in a biological sample from a patient; and correlating the level of gene expression with a score from a rating scale such as Hoehn and Yahr scale (H&Y), Modified Schwab and England Activities of Daily Living (Modified Schwab and England), and Unified Parkinson Disease Rating Scale (UPDRS), thereby prognosing the patient with slow or rapid progression of the disease.

Further described herein is a method for prognosing a patient presenting with Parkinson's Disease-related cognitive decline, which includes: determining a level of expression of at least one gene such as HSPA8 and SKP1a in a biological sample from a patient; and correlating the level of gene expression with a score from a rating scale such as Montreal Cognitive Assessment (MoCA), Mini Mental State Examination (MMSE), Hopkins Verbal Learning Test (HVLT) and University of Pennsylvania Smell Identification Test (UPSIT), thereby prognosing the patient with PD-related cognitive decline.

Further described herein is a method for prognosing a patient presenting with Parkinson's Disease-related symptoms, which includes: determining a level of expression of at least one gene such as LAMB2 and SKP1a in a biological sample from a patient; and correlating the patient's level of gene expression with unfavorable results of MDS UPDRS, thereby prognosing the patient with rapid progression of the PD-related Dyskinesia.

### III. Methods for Prognosis and Treatment of Parkinson's Disease

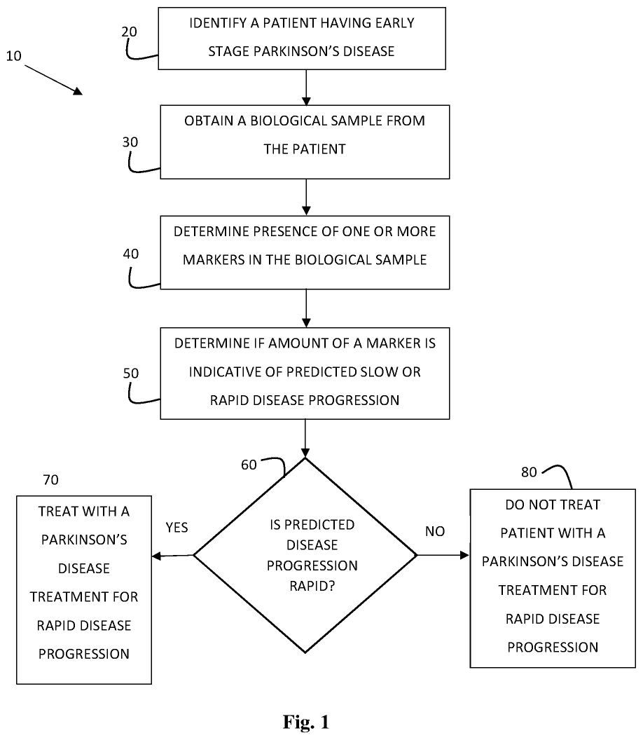

Reference is now made to FIG. 1 which depicts a flow diagram showing a method 10 for determining the prognosis of a subject which in turn dictates the course of treatment of a patient suffering from early stage PD. Flow diagram also depicts a method for optimizing treatment and/or a method for determining an optimal treatment.

Method 10 comprises block 20, comprising identification of a patient having early stage PD. Early stage PD patients may be patients who have been diagnosed with PD for no longer than three years. Optionally, early stage PD patients may be patients who have been diagnosed with PD for no longer than two years.

Early stage PD patients may be patients identified as having PD in a stage 2 or below according to the Hoehn and Yahr scale. Stage 2 of the Hoehn and Yahr scale is defined as bilateral or midline involvement without impairment of balance. Hoehn and Yahr scale stages are shown in Table 1 below.

TABLE 1

| Stage | Description |
|---|---|
| 1 | Unilateral involvement only usually with minimal or no functional |
| | disability |
| 2 | Bilateral or midline involvement without impairment of balance |
| 3 | Bilateral disease: mild to moderate disability with impaired |
| | postural reflexes; physically independent |
| 4 | Severely disabling disease; still able to walk or stand unassisted |
| 5 | Confinement to bed or wheelchair unless aided |

Early stage PD patients may be naïve patients who have not yet received Parkinson's disease treatment.

Early stage PD patients may be patients having a Modified Schwab and England Activities of Daily Living Scale (MSEADL) of greater than 70%. MSEADL scale stages are shown in Table 2 below:

TABLE 2

| Stage | Description |
|---|---|
| 100% | Completely independent. Able to do all chores without |
| | slowness, difficulty or impairment. Essentially normal. |
| | Unaware of any difficulty. |
| 90% | Completely independent. Able to do all chores with some |
| | degree of slowness, difficulty and impairment. Might |
| | take twice as long. Beginning to be aware of difficulty. |
| 80% | Completely independent in most chores. Takes twice as long. |
| | Conscious of difficulty and slowness. |
| 70% | Not completely independent. More difficulty with some chores. |
| | Three to four times as long in some. Must spend a large part |
| | of the day with chores. |
| 60% | Some dependency. Can do most chores, but exceedingly slowly |
| | and with much effort. Errors; some impossible. |
| 50% | More dependent. Help with half, slower, et cetera. Difficulty |
| | with everything. |
| 40% | Very dependent. Can assist with all chores, but few alone. |
| 30% | With effort, now and then does a few chores alone or begins |
| | alone. Much help needed. |
| 20% | Nothing alone. Can be a slight help with some chores. Severe |
| | invalid. |
| 10% | Total dependent, helpless. Complete invalid. |
| 0% | Vegetative functions such as swallowing, bladder and bowel |
| | functions are not functioning. Bed-ridden. |

Early stage PD patients may be patients having Montreal Cognitive Assessment (MoCA) scores of 26 to 29 with an average of 27. A score of 26 or over is considered to be normal. Progression to less than 24 within two years of diagnosis indicates rapid disease progression. In early Parkinson disease, when cognitive deficits occur, they are subtle and mild and the patients usually perform in the normal range on the widely used Mini Mental State Examination (MMSE). The Montreal Cognitive Assessment (MoCA) is a rapid screening instrument like the MMSE but was developed to be more sensitive to patients presenting with mild cognitive complaints. It assesses short term and working memory, visuospatial abilities, executive function, attention, concentration, language and orientation. The total score ranges from 0 to 30.

Early stage PD patients may be patients having Hopkins Verbal Learning Test (HVLT) scores of 39 to 54 with an average of 46. A decrease to less than 30 within two years of diagnosis indicates rapid disease progression.

HVLT is a screening test relating to memory impairment.

Method 10 comprises block 30, comprising obtaining a biological sample from the patient. The biological sample may comprise blood.

Method 10 comprises block 40, comprising determining the presence of one or more markers in the biological sample. The marker may be one or more RNA molecule.

When the biological sample is blood, the blood may be drawn into a receptacle from the patient, and then the RNA may be stabilized. Total RNA may be extracted from the blood sample using methods known in the art. The concertation and level of degradation of the RNA in the sample is evaluated.

A fixed amount of RNA may be used as a template for synthesis of complementary DNA (cDNA) to the sample RNA using reverse transcription enzyme and buffer and reagent.

The genes used as markers may be any one or a combination of those genes detailed in table 3A below. Levels of marker gene expression may be compared to levels of reference gene expression. The genes used as reference genes may include any genes having expression levels which are relatively unaltered by disease progression Optionally, the reference genes may be those detailed in table 3B below.

TABLE 3A
Genes used as markers

| | Genbank Accession | |
|---|---|---|
| Marker genes | transcript number | Symbol |
| S-phase kinase-associated | NM_006930 | SKP1A |
| protein 1A | | |
| Huntingtin interacting | NM_005339, | HIP2 or |
| protein-2 | NM_001111113 | UBE2K |
| Aldehyde dehydrogenase family | NM_000689 | ALDH1A1 |
| 1 subfamily A1 | | |
| Proteasome (prosome, macropain) | NM_006503 | PSMC4 |
| 26 S subunit, ATPase 4 | | |
| Heat shock 70-kDa protein 8 | NM_006597, | HSPA8 |
| | NM_153201 | |
| Laminin subunit beta 2 | NM_002292.3 | LAMB2 |

TABLE 3B
Reference genes used

| | Genbank Accession | |
|---|---|---|
| Reference genes | transcript number | Symbol |
| Phosphoglycerate kinase 1 | NM_000291 | PGK1 |
| Glyceraldehyde-3-phosphate | NM_002046 | GAPDH |
| dehydrogenase | | |

Optionally, the relative quantity of the marker genes in the sample relative to amount of at least one reference gene is determined. The relative quantity can determined using for example Real Time quantitative Polymerase Chain Reaction (qPCR), or next generation sequencing (NGS), or DNA micro array.

Method 10 comprises block 50, comprising determining if an amount of a marker is indicative of predicted slow or rapid disease progression, and thus providing a prognosis of the disease. The genes listed can be considered as biomarkers, in which the biomarker expression levels predicts faster rate of disease progression effectively determining the prognosis of the disease.

Disease progression may be progression of a physical symptom of disease. Disease progression may be progression of a cognitive symptom of disease.

Rapid disease progression in Parkinson may be associated with increased difficulties in activities of daily life, such as: more intense tremor, more difficulties standing up, more difficulties to eat and pick up utensils, less capability to walk independently with short steps and sudden stops (freezing spells), and offs (stopping while walking which last minutes to hours). In general, rapid disease progression, when presented to a physician, is accompanied by an increase in the patient's medication. Other symptoms accompanied with rapid disease progression include: decreased equilibrium while walking, leading to frequent falls. Other symptoms accompanied with rapid disease progression include voice changing to a lower pitch with more sialorrhea (saliva drooling) and less strength. This change in voice may lead to difficulty in understanding the patient's speech.

There are psychological ramifications of rapid disease progression in PD. Patients may become apathetic and reluctant to interact with family and friends. Patients may become depressed. Cognitive limitations (confusional episodes) and visual hallucinations may also be observed. In addition patients may develop delusions with paranoidal content. Patients may sometimes become aggressive with difficulties to restrain themselves with impulse control features.

Over time, as disease progresses, the patient may need to use a cane or a walker and gradually become chair ridden due to frequent falls. The most common causes of death in PD patients are Bronchopneumonia due to swallowing events; sepsis due to fall injuries with bone broken episodes; and urinary sepsis. The process of rapid disease progression may progress about 7 to 10 years from initial PD diagnosis.

If a patient has slow disease progression, the patient may remain with symptoms of such as tremor, but will stay generally independent. The course of the disease may last about 10-15 years before the symptoms worsen.

Though the exact cause of dyskinesia is uncertain, but most agree that it is related to the long-term use of certain medications, including levodopa. It is thought that an increased sensitivity to dopamine in the brain as a result of levodopa, combined with the natural progression of Parkinson's, gives rise to dyskinesia.

Optionally, disease progression may be based on physical progression of disease, optionally as determined by MSEADL. Optionally, disease progression may be based on cognitive progression of disease, optionally determined by MoCA.

The prognosis may be based solely on biomarker gene expression amount. Alternatively, additional factors may be taken into consideration to determine if marker amount is indicative of slow or rapid disease progression. Additional factors taken into consideration in conjunction with the marker amount include: age of patient, sex of patient, smell acuity of patient.

Prognosis may be determined based on amount of marker genes ALDH1A1, LAMB2, UBE2K AND SKP1a. Prognosis may be further determined using the aforementioned marker genes in combination with age of the patient.

Prognosis may be determined based on amount of marker genes HSPA8 and SKP1a. Prognosis may be further determined using the aforementioned marker genes in combination with a smell acuity of a patient as determined by the University of Pennsylvania Smell Identification Test (UPSIT) and/or age of the patient.

The amount, or level of marker genes may be determined (i.e. detected) using qPCR or similar methods. Using qPCR, the number of cycles required for a fluorescent signal to cross a certain threshold (also known as “cycle threshold” or “CT” value) may be obtained for a marker gene and for a reference gene. A Delta CT (ΔCT) value may be then obtained by subtracting the CT of a reference gene from CT of a target gene. When ΔCT values are obtained for a target sample and a reference sample, the ΔCT of the reference sample may be subtracted from the ΔCT of the target sample to obtain a delta delta CT (ΔΔCT or ddCT) value. The obtained ΔΔCT value is reflective of the fold-change of target gene expression in a target sample relative to a reference sample, normalized to a reference gene. The reference sample may be a mix of the synthetic mRNA segments or mix of extracted human RNA. The ΔΔCT value inversely relates to the level of gene expression in the sample.

Once ΔΔCT is obtained for a marker gene, marker genes may be weighted using an appropriate coefficient. The sum of the weighted values for the marker genes may be known as a classifier. The classifier may then be converted into a predicted probability using the equation=1/(1+EXP(−1*Classifier)).

A receiver operating characteristic (ROC) curve may be formed based on historical data obtained from patients analyzed for marker amounts at early stage, and who have proceeded over time to exhibit either slow disease progression or to rapid disease progression.

Once an ROC curve has been obtained, a predicted prognosis for a patient may be compared to a value along the ROC curve to assess likelihood of rapid disease progression. Optionally, the patient is considered to have a high likelihood of rapid disease progression if likelihood of a patient of having rapid disease progression is above 60%, 70%, 75%, 80%, 85%, 90% or 95%.

Method 10 comprises block 70, comprising treating a patient with a Parkinson's disease treatment for rapid disease progression if patient exhibits a high likelihood of rapid disease progression, in which an unfavorable prognosis is associated.

The disease treatment may be selected from the group consisting of: levodopa treatment, carbidopa-levodopa; and deep brain stimulation treatment.

In a patient wherein the prognosis shows a rapid disease progression is associated with cognitive decline, such as behavioral symptoms, the treatment may be an atypical antipsychotic. The atypical antipsychotic may be clozapine. In a patient wherein rapid disease progression is associated with cognitive decline, such as dementia, the treatment may be a cholinesterase inhibitor or a glutamine inhibitor. An exemplary cholinesterase inhibitor may be donepezil, rivastigmine or galantamine. An exemplary glutamine inhibitor may be memantine.

In a patient wherein the prognosis indicates a rapid disease progression is associated with dyskinesia, the treatment may be lowering dose of levodopa, administering extended release levodopa, or continuous infusion levodopa. The treatment may be amantadine. The treatment may be deep brain stimulation.

Method 10 comprises block 80, comprising not treating a patient with a Parkinson's disease treatment for rapid disease progression if patient does not exhibit a high likelihood of rapid disease progression. Block 80 may comprise not treating a patient with levodopa treatment, carbidopa-levodopa, and deep brain stimulation treatment. Block 80 may comprise treating a patient with a MAO-B inhibitor, and/or a dopamine agonist.

The following examples are provided to illustrate certain particular features and/or embodiments. These examples should not be construed to limit the disclosure to the particular features or embodiments described.

### EXAMPLES

### Example 1—Analysis of Early Stage PD Patients

In a cohort of PD patients from the “Parkinson's Progression Markers Initiative” (PPMI) study, the relative gene expression levels of SKP1a, UBE2K, ALDH1A1, PSMC4, HSPA8 and LAMB2 were measured in baseline blood samples by real-time quantitative PCR. PD patients were up to two years from diagnosis, Hoehn and Yahr (H&Y) stage I or II and PD treatment naïve. Blood samples were collected every 3 months for 3 years.

Gene expression levels were screened in available baseline blood samples from PD patients (n=279) whose mean age was 59.4 years, having the range between 30 to 83 years, 96 females and 183 males. The statistical analysis was performed on PD patients with recorded Modified Schwab and England Activities of Daily Living scores from their third year study visit (n=241 of 279), PD patients' mean age was 53.9 years (Range was 31 to 73 years, 96 female and 183 male. The patients were not prescribed PD medicine at the time of baseline blood collection, and were not expected to require PD medication within at least six months from baseline. The patients were enrolled in the study if they had at least two of the following: resting tremor, bradykinesia, rigidity (must have either resting tremor or bradykinesia); Or either asymmetric resting tremor or asymmetric bradykinesia.

Patients were enrolled if diagnosis of PD was for two or fewer years, and a Hoehn and Yahr (H&Y) score of stage I or II at baseline. Dopamine (DA) transporter deficit was confirmed using DaTscan™, a technology using ioflupane iodine-123 injection visualized by single photon emission computed tomography. In sites where DaTscan™ was not available, patients were analyzed for VMAT-2 deficit by using positron emission tomography (PET) scan. The study was approved by the institutional review board at each site, and participants provided written informed consent.

Blood was collected from the patients in blood RNA tubes (PAXgene tubes PreAnalytiX, Switzerland) and mRNA was extracted by the PPMI study team, according to manufacturer's recommendations. RNA samples with a concentration of at least 20 ng/μl, and RNA Integrity Number (RIN) >5.0 were selected for the study. RNA samples (1 μg RNA, variable volume and concentration) were shipped frozen (70° C.) Researchers were blinded to the clinical identity and time of collection of the RNA samples. Relative expression of S-phase kinase-associated protein 1A (SKP1a), Huntingtin interacting protein-2/Ubiquitin Conjugating Enzyme E2K (HIP2/UBE2K), Aldehyde dehydrogenase family 1 subfamily A1 (ALDH1A1), Proteasome (prosome, macropain) 26 S subunit, ATPase 4 (PSMC4), Heat shock 70-kDa protein 8 (HSPA8) and laminin beta2 (LAMB2) were measured. Briefly, mRNA was reverse-transcribed to cDNA. The cDNA was diluted to the testing concentration. Real-time quantitative PCR (RT-qPCR) was performed in a 96-well format, 25 μL total reaction volume using RT-qPCR master mix with SYBR Green florescence. A single PCR reaction was performed in each well. In order to avoid any operator biases or inaccuracies, a dedicated automatic pipetting system (EZmate™ 400 by ARIS Biotech, USA) was used for mixing the reagents (primers, master mix, cDNA samples, positive controls, calibrator, and water) and dispensing them onto the PCR plates. In each plate, four cDNA samples for the expression of six target genes and two reference genes were tested. Measurements of the samples and calibrator were performed in duplicates. Each plate included negative control (no template), specific positive control of each gene (comprised of synthetic amplicon in a predefined concentration), calibrator/reference sample comprised of a mix of the synthetic amplicons for normalize plate to plate variations and cDNA samples of four patients.

Following plate preparation, the assay was performed using the StepOne™ PCR machine (ThermoFisher, USA). Each run included amplification cycles and melt curve analysis for quality control. Relative expression of each of the target genes in each sample was calculated by the ddCT method using the StepOne™ dedicated software. All run parameters were exported to the study's database. Quality assurance parameters were calculated and evaluated regarding precision between duplicates dCt SD (<0.5), absolute Ct values of controls (positive, negative and calibrator) within a predefined range, and absence of multiple peaks in the melt curve. If results did not match QC criteria, the sample was re-run. The delta delta CT values inversely relate to the level of gene expression in the sample.

Statistical analysis was performed using MedCalc Statistical Software version 16.2.1 (MedCalc Software bvba, Ostend, Belgium; https://www.medcalc.org; 2016). Multivariable logistic regression analysis was performed for determining the composition of the predictive classifier algorithm (CL-Prognosis); Variables were kept in the model if their P Value was less than 0.15 and the variable improved the model performance. The discriminant performance and cutoff values of CL-Prediction were determined by receiver operating characteristic (ROC) curve. Kaplan Meier and hazard ratios were used for assessing the prediction ability CL-Prediction classifier. Chi-square test was used to test proportional differences. Uncertainty of results was expressed by 95% confidence intervals P-values of less than 0.05 were considered to be statistically significant. CL-Prognosis classifier enables possible prognosis of the PD progression.

### Example 2—Determination of PD Prognosis from Gene Expression Analysis

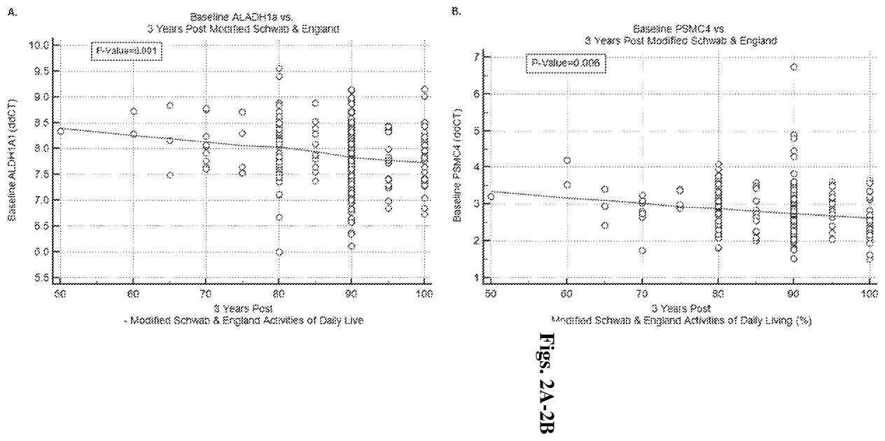

Patients' blood samples were analyzed as in Example 1. The association of baseline gene expression levels in blood samples and 3 years post-baseline Modified Schwab and England Activities of Daily Living Scale (Modified Schwab and England) were tested by Spearman's rho rank correlation. Baseline ddCT levels of genes ALDH1A1 (P-Value=0.001, rho=−0.208, 95% CI −0.326 to −0.0839) and PSMC4 (P-Value=0.006, rho=−0.178, 95% CI −0.298 to −0.0522) significantly decreased while the Modified Schwab and England Scale increased at 3 years after blood sample collection (FIG. 2). A similar non-significant trend was observed for LAMB2 (P-Value=0.075, rho=−0.115, 95% CI −0.238 to 0.0116). Thus indicating that an unfavorable prognosis will correlate to a decrease in baseline expression of the genes and a decrease in the Modified Schwab and England Scale.

Logistic Regression analyses were performed in order to construct a classifier comprised of the gene expression baseline values for identifying PD patients who showed rapid disease progression as shown by Modified Schwab and England ≤70%, the initial score when the PD patient is not completely independent, by 3 years post-baseline. Variables with P-values<0.15 were included in subsequent models until reaching a model with all variables having a P-value<0.15. The model included ALDH1A1, LAMB2, SKP1a, UBE2K and age (Table 4). The coefficients of the model (Model 3) were used to build the classifier's algorithm (CL-Prediction) to calculate the predictive probability (PP) values, indicating rapid prognosis of the disease.

TABLE 4
Logistic regression model to determine Prognosis

| Baseline | Coeffi- | Std. | | OR | |
|---|---|---|---|---|---|
| Variables | cient | Error | Wald | (95% CI) | P |
| ALDH1A1 | 1.5479 | 0.57909 | 7.1447 | 4.70 | 0.0075 |
| (ddCT) | | | | (1.51 to | |
| | | | | 14.63) | |
| LAMB2 (ddCT) | −0.91861 | 0.39914 | 5.2967 | 0.40 | 0.0214 |
| | | | | (0.18 to | |
| | | | | 0.88) | |
| UBE2K (ddCT) | −0.21651 | 0.14399 | 2.2611 | 0.81 | 0.1327 |
| | | | | (0.61 to | |
| | | | | 1.07) | |
| SKP1a (ddCT) | 1.15002 | 0.68262 | 2.8382 | 3.16 | 0.092 |
| | | | | (0.83 to | |
| | | | | 12.03) | |
| Age (years) | 0.11518 | 0.038137 | 9.1208 | 1.122 | 0.0025 |
| | | | | (1.04 to | |
| | | | | 1.21) | |
| Constant | −12.4435 | 5.59174 | 4.9521 | | 0.0261 |

| Overall Model | 0.0004 |
|---|---|
| Fit | |
| ROC of | 0.852 |
| Algorithm | (0.800 to 0.894) |

The predictive classifier algorithm based on constant and coefficients in Table 4 was equal to: (1.5479*ALDH1a)+(−0.91861*LAMB2)+(−0.21651*UBE2K)+(1.15002*SKP1a)+(0.11518*Age)−12.4435.

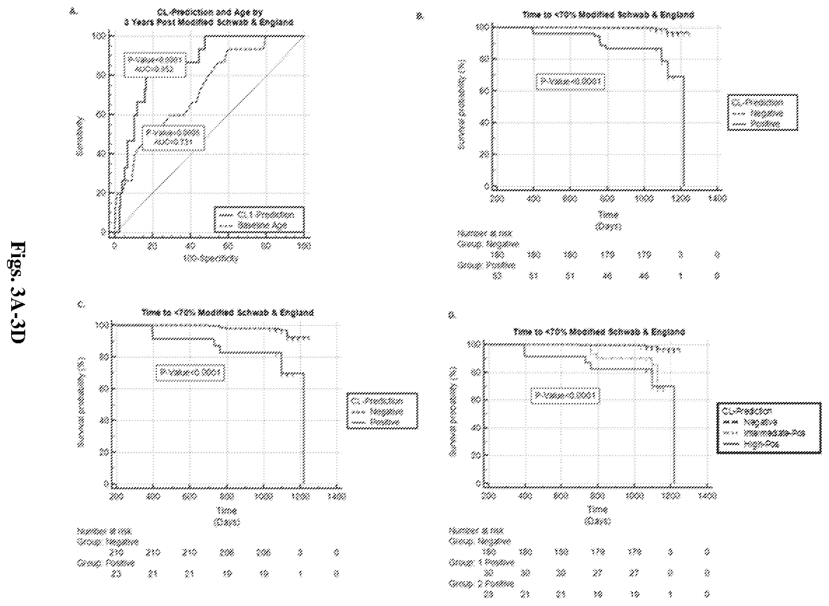

Receive operator characteristics (ROC) curve was used to determine cutoff values for positive and negative model 3 Classifier Prediction (CL-prediction) predicted probability (PP) values (FIG. 3A). The cutoff at the Youden Index, which is J=max (sensitivity-c+specificity-c−1, graphically J is the maximum vertical distance between the ROC curve and the diagonal (specificity=81.7, sensitivity=80.0).

Time-to-event analyses were performed for testing the prognostic ability of CL-Prediction. Time from baseline to the endpoint was calculated for each patient, i.e. if the patient reached ≤70% Modified Schwab and England by the 2nd year visit then time to the endpoint was calculated as time (days) from baseline to the 2nd year visit. For patients that did not reach the endpoint the last reported visit was included in the analysis. Patients who reached endpoint, but then in the subsequent Visit improved to above the endpoint were not included in the analysis (n=5).

PD patients positive for CL-Prediction Classifier (Youden Index cutoff) had faster time to ≤70% Modified Schwab and England (risk to more severe PD) than patients who were negative for CL-Prediction (P Value<0.0001, Kaplan Meir curve); the positive hazard ratio (HR+) of reaching the endpoint was 13.6 (95% CI, 4.1-45.4), negative hazard ratio (HR−) was 0.074 (95% CI, 0.022-0.25). Median time to endpoint of patients who were positive for the marker was 1218 days (95% CI, 1218 to −). Patients who were negative for the marker did not reach the median time to endpoint (FIG. 3B).

An additional cutoff (high specificity) for positive CL-Prediction was investigated at the PP value corresponding to a specificity of 93.3%, sensitivity of 46.7% and positive likelihood ratio (LR+) 7.0. At the high specificity cutoff, PD patients positive for CL-Prediction had faster time to ≤70% Modified Schwab and England than patients who were negative for CL-Prediction (P-Value<0.0001, Kaplan Meir curve); the HR+ of reaching the endpoint was 7.42 (95% CI, 1.43-38.5), HR− was 0.13 (95% CI, 0.026-0.70). Median time to endpoint of patients who were positive for the classifier was 1218 days (95% CI, 1096 to −). Patients who were negative for the classifier did not reach the median time to endpoint (FIG. 3C).

In a time-to-event analysis using both the Youden Index (Intermediate-Pos) and high specificity (High-Pos) cutoffs the CL-Prediction classifier was able to predict PD patients who progressed faster to ≤70% Modified Schwab and England (P-Value<0.0001). The HR+at the High-Pos cutoff was 17.08 (95% CI, 3.24-89.89), the HR+at the Intermediate-Pos cutoff was 10.55 (95% CI, 2.22-50.12) and HR− was 0.059 (95% CI, 0.11-0.31) (FIG. 3D). The median time to progression to ≤70% Modified Schwab and England of High-Pos was 1218 days (95% CI 1096 to −).

Cross-sectional analysis showed the linear increase [P-Value<0.0001, Chi-squared (trend)] in risk to more severe PD (≤70% Modified Schwab and England) by three years post diagnosis (baseline visit): High-Pos patients was 30.4% (7/23), Intermediate-Pos was 16.7% (5/30) and Negative was 1.7% (3/180).

CL-prediction algorithm was successful in determining the prognosis of patients and discerning between either rapid or slow progression of PD as determined by Modified Schwab and England Score, as shown. In addition the CL-prediction algorithm was successful in discerning between rapid and slow progression of PD as determined by UPDRS I, II, III and IV.

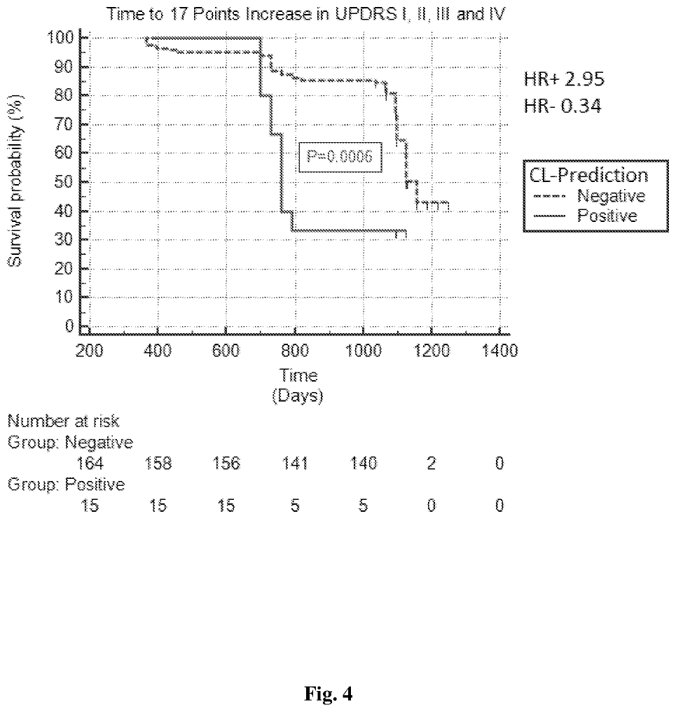

An increase of 17 points between baseline levels and 3 years after baseline, in UPDRS could be predicted using the CL-prediction algorithm. At High-Pos, positive hazard ratio (HR+) was 2.95 (95% CI, 1.04-8.36), negative hazard ratio (HR−) was 0.34 (95% CI, 0.12 to 0.96). Median time to increase of 17 points in positive patient group was 761 days (96% CI 730 to 793) and in the negative patient group=1156 days (95% CI, 1126-1156). FIG. 4 shows the Kaplan-Meir curves of CL-Prediction for determining the prognosis of PD patients who display the characteristics of a faster disease progression as shown by time to increase of UPDRS score by 17 points.

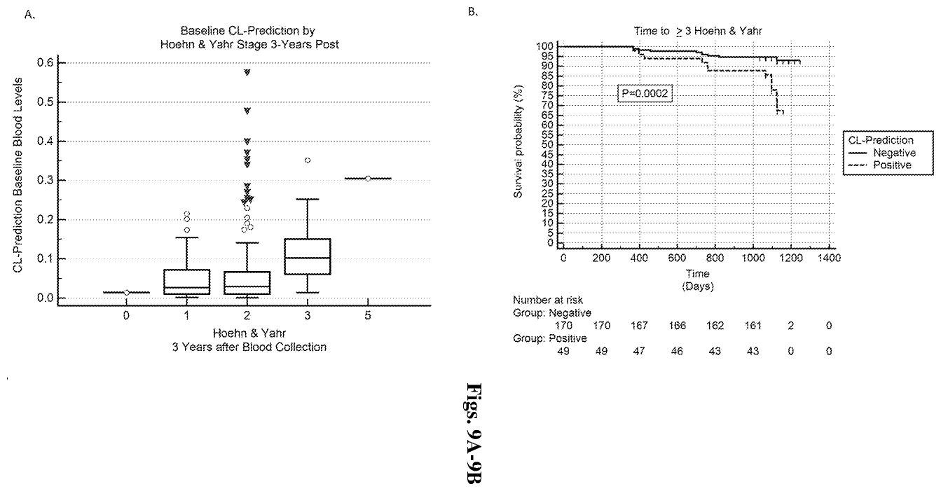

Patients' blood samples were analyzed as in Example 1. A similar analysis to the Modified Schwab and England was performed showing the CL-Prediction on a Hoehn and Yahr (H&Y) Scale (FIG. 9). H&Y stage at 3 years-post baseline shows CL-Prediction PP baseline values were significantly higher (P-Value=0.0001) in baseline blood of PD patients who reached H&Y stage≥3 (median 0.351 PP value, 95% CI 0.0621 to 0.174) than PD patients whose H&Y stage<3 (median 0.029 PP value, 95% CI 0.020 to 0.036) at 3 years post-baseline (average days±SD was 1106±29 days) (FIG. 9A). Additionally, FIG. 9B shows Kaplan-Meir curves predicting time to H&Y stage≥3 using the Youden Index Cutoff. CL-Prediction significantly distinguished patients (P-Value=0.0002) with faster time to H&Y stage≥3 using Cutoff-1 with an HR+ of 4.3 (95% CI, 1.6 11.6) and HR− of 0.24 (95% CI, 0.085-0.65). Thus indicating that an unfavorable prognosis will correlate with a decrease in baseline expression of the genes and an increase in the H&Y Scale.

This example shows that prognosis of a patient suspected of PD may be predicted to a high level of certainty, based on analysis of gene expression in a biological sample from a patient.

### Example 3: Prognosis of PD Patient Cognitive Decline

Patients' blood samples were analyzed as in Example 1. Patients were tested for MoCA score at baseline and at 3 years post-baseline. A correlation between gene expression blood levels from 1-year post baseline to cognitive scale as defined by MoCA 3-years post baseline was found. Gene expression of HSPA8 (P=0.027, rho=−0.252) correlated with HVLT score. Gene expression of HSPA8 (P=0.029, rho=−0.248) and SKP1a expression (P=0.041, rho=0.232) correlated with MoCA scale and associated with <24 MoCA vs. >24 MoCA, HSPA8 (P=0.0003), SKP1a (P=0.003) and PSMC4 expression (P=0.015) as tested by Mann-Whitney. Indicating that a cognitive decline will correlate with an increase in HSPA8 and SKP1a expression level.

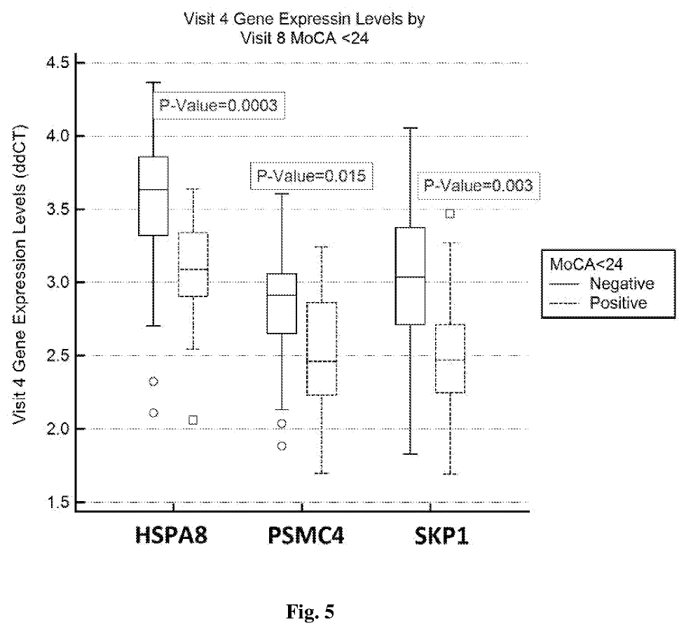

FIG. 5 shows box plots of HSPA8, PSMC4 and SKP1a ddCT levels at Visit 4 (1 year post-baseline) of PD patients by MoCA below 24 vs. equal and above 24. The central box represents 25 to 75 percentile, the middle line represents the median, lines extends to the maximum and minimum values, outliers are depicted as blue circles (than the lower quartile minus 1.5 times the quartile range. Gene ΔΔCT levels are lower for each of the genes, indicating an increase in gene expression levels, in patients having MoCA scores lower than 24, indicating a correlation between each of the genes' expression and cognitive decline.

Logistic regression analyses were performed in order to construct a classifier (cognitive predictive classifier) comprised of the gene expression values (1 year after baseline) for identifying PD patients who showed rapid cognitive decline as defined by MoCA score decrease to lower than 24 at 3 years post-baseline. In addition to gene expression values, age of patient and UPSIT score were used. An algorithm comprised of HSPA8, SKP1a, age of patient and UPSIT score, created by logistic regression, showed high diagnostic performance as seen in ROC curve, AUC=0.915 (0.821 to 0.969), P<0.0001). Using cutoff based on the ROC curve (83% specificity, 86% sensitivity), the classifier predicted high risk to progression of <24 MoCA score by three years of BL. Kaplan Meier analysis resulted in HR+=19.3 (95% CI, 5.6-66.3), and HR−=0.052 (95% CI, 0.015-0.18) for progression to <24 MoCA within three years of diagnosis. The proportion of patients positive for the cognitive prognosis algorithm were 85.7% (12/14) for high risk and 13.2% (7/53) for low risk (P value<0.0001). The algorithm is detailed in Table 5 below:

TABLE 5
Parameters of Cognitive Prognosis in PD patients

| | Coeffi- | Std. | | OR | |
|---|---|---|---|---|---|
| Variables | cient | Error | Wald | (95% CI) | P |
| HSPA8 (ddCT)- | −2.39361 | 1.00419 | 5.6816 | 0.091 | 0.0171 |
| V04 | | | | (0.013- | |
| | | | | 0.65) | |
| SKP1a (ddCT)- | −1.27186 | 0.80674 | 2.4855 | 0.28 | 0.1149 |
| V04 | | | | (0.057 to | |
| | | | | 1.36) | |
| Age (yrs.) | 0.15828 | 0.070381 | 5.0577 | 1.17 | 0.0245 |
| | | | | (0.795- | |
| | | | | 1.35) | |
| UPSIT (Score) | −0.11884 | 0.056685 | 4.3957 | 0.888 | 0.036 |
| | | | | (0.795- | |
| | | | | 0.992/) | |
| Constant | 1.54744 | 4.7284 | 0.1071 | | 0.7435 |

| Overall Model | <0.0001 |
|---|---|
| Fit | |
| ROC of | 0.896 |
| Algorithm | (0.806-0954) |

The cognitive prognosis classifier algorithm based on constant and coefficients in Table 5 was equal to (−2.39361*HSPA8)+(−1.27186*SKP1a)+(0.15828*Age)+(−0.11884*UPSIT)+1.54744.

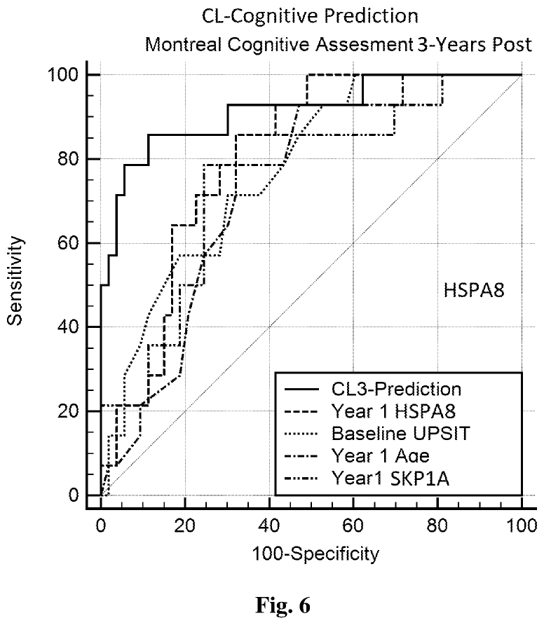

FIG. 6 shows receiver operator characteristics (ROC) curves for differentiating PD patients who progressed to <24 MoCA within three years of diagnosis by 3-years post baseline. The solid line is the CL-Cognitive Prognosis algorithm comprised of expression levels (ddCT) of HSPA8 and SKP1a 1-Year post baseline, age 1-Year post baseline (years) and UPSIT at blood collection; CL-Cognitive Prognosis AUC is 0.915 (0.821 to 0.969), P value<0.0001. Other curves are shown for comparison and were significant. This indicates that the prognosis determined by the cognitive classifier is more accurate and effective in determining which patients will have rapid cognitive decline than any of the single genes, age and UPSIT scores.

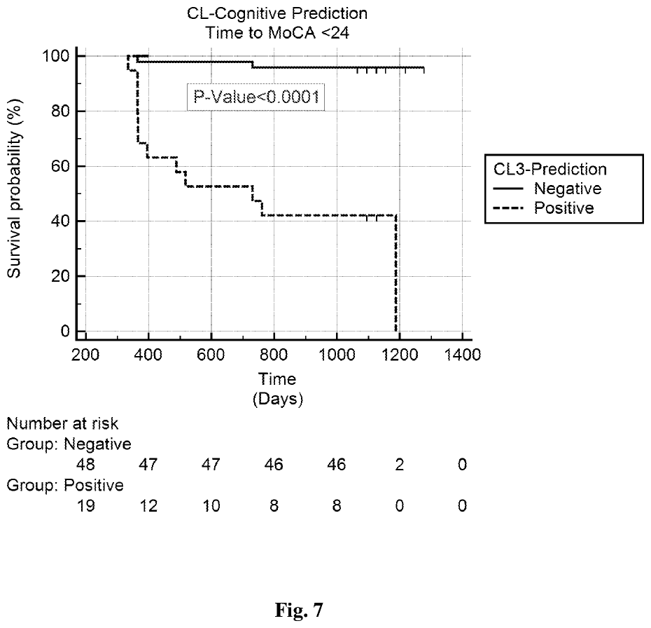

FIG. 7 shows Kaplan-Meir curve of CL-Cognitive Prediction for predicting PD patients who have faster time to <24 MoCA. The HR+ of reaching the endpoint was 19.33 (95% CI, 5.64-66.28), HR− was 0.052 (95% CI, 0.015-0.18). Median time to endpoint was 731 days (95% CI, 366 to 1188). Patients who were negative for the marker did not reach the median time to endpoint. P-Value was <0.0001.

Since not all PD patients have available UPSIT test results, an algorithm comprised of HSPA8, SKP1a and Age, without UPSIT score, was tested by Kaplan Meier resulting in HR+9.81 (95% CI, 3.02-31.8], and HR− 0.10 (95% CI, 0.03-0.1833) for progression to <24 MoCA within three years of diagnosis. There were 3/46 (6.5%) patients who were negative for the classifier who reached <24 MoCA and 11/21 (52%) of patients who were positive for the classifier who reached <24 MoCA by year 3. Similar results with the same algorithm was found for Hopkins Verbal Learning Test Total Score progression to <30 points by 3-years post baseline (P<0.0001, AUC=0.918, 95% CI, 0.811 to 0.975, ROC curve analysis). The algorithm is described in detail below in Table 6:

TABLE 6
Restricted Parameters for Cognitive Prognosis in PD patients

| | Coeffi- | Std. | | OR | |
|---|---|---|---|---|---|
| Variables | cient | Error | Wald | (95% CI) | P |
| HSPA8 (ddCT)- | −2.36482 | 0.98775 | 5.7319 | 0.094 | 0.0167 |
| V04 | | | | (0.014- | |
| | | | | 0.65) | |
| SKP1a (ddCT)- | −1.39981 | 0.82412 | 2.8851 | 0.247 | 0.0894 |
| V04 | | | | (0.049 to | |
| | | | | 1.24) | |
| Age | 0.18071 | 0.066257 | 7.4391 | 1.198 | 0.0064 |
| | | | | (1.05- | |
| | | | | 1.36) | |
| Constant | −1.8556 | 3.9822 | 0.2171 | | 0.6412 |

| | |
|---|---|
| ROC of | 0.874 |
| Algorithm | (0.779-0.938) |

The cognitive predictive classifier algorithm based on constant and coefficients in Table 6 was equal to (−2.36482*HSPA8)+(−1.39981*SKP1a)+(−0.18071*Age)−1.8556.

This example shows that the prognosis of a PD patient's cognitive decline, whether rapid or slow, may be predicted at diagnosis or within a year of diagnosis to a high level of certainty, based on analysis of gene expression in a biological sample from a patient.

### Example 4: Prognosis for Development of Dyskinesia in PD Patients Based on Gene Expression

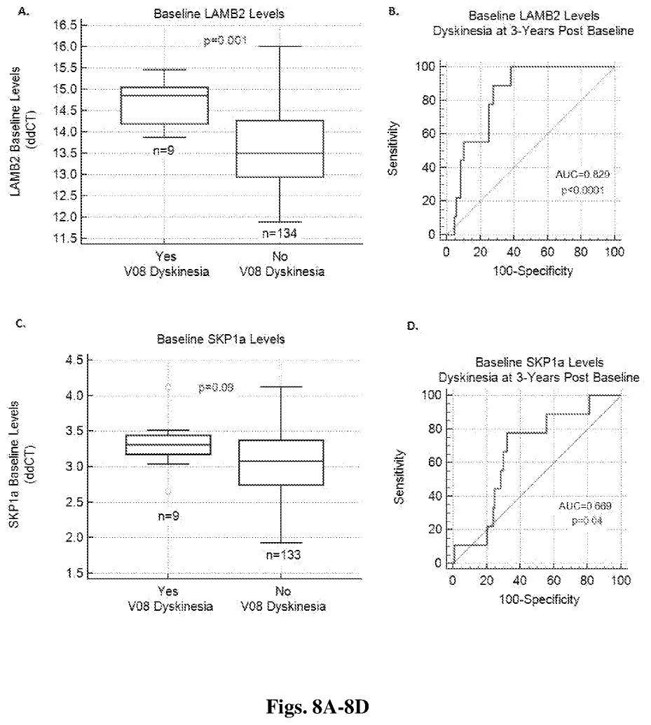

Further study revealed an association of the expression level of LAMB2 at baseline with PD patients succumbing to dyskinesia by 3-Years post Baseline visit as reported in MDS UPDRS assessed by clinical exam. PD patients who developed earlier dyskinesia had higher ddCT levels, indicating decrease in gene expression (14.85, 95% CI 14.16 to 15.26 ddCT, n=9) of LAMB2 than PD patients who did not succumb (13.50, 95% CI, 13.30 to 13.72 ddCT, n=134) to Dyskinesia by 3-years post baseline (p=0.001, Mann-Whitney). This is shown in FIG. 8A. A similar trend was seen for SKP1a expression (p=0.09), (FIG. 8C) which became significant without low outlier (p=0.03). The ability to differentiate between patients who were likely to have Dyskinesia at 3-Years post Baseline was also evident in the LAMB2 ROC curve analysis (AUC 0.829 95% CI 0.747 to 0.887, p<0.0001) as shown in FIG. 8B. Crossing of the LAMB2 curve occurred at ˜95% specificity, but a clear ability to detect early dyskinesia is obvious from <95% specificity. SKP1a expression was also significant for detecting early dyskinesia by ROC analysis (AUC 0.669 95% CI 0.585 to 0.746, p=0.04), but only from <76% specificity, as shown in FIG. 8D. Exclusion of low outlier of SKP1a levels lead to better performance (AUC 0.729 95% CI 0.648 to 0.801). This indicates that an unfavorable MDS UPDRS score and a decreased expression of LAMB2 and SKP1a correlate to the development of dyskinesia. The clinical information was taken at post-medication. No association was found between LAMB2 or SKP1a levels at Baseline or 3-Years post-Baseline and use of L-DOPA treatment at 3-Years post Baseline.

This example shows that a patient's prognosis in terms of dyskinesia, whether rapid or slow, may be predicted at diagnosis or within a year of diagnosis to a high level of certainty, based on analysis of gene expression in a biological sample from a patient.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method for prognosing a patient presenting with early stage Parkinson's Disease (PD) symptoms, comprising:
determining a level of expression of at least one gene selected from the group consisting of SKP1a, UBE2K, ALDH1A1, PSMC4, HSPA8 and LAMB2 in a biological sample from a patient, and
correlating the level of gene expression with a Parkinson's Disease rating scale, thereby prognosing the patient with slow or rapid progression of the symptoms or disease.

2.-17. (canceled)

18. A method of reducing Parkinson's disease symptoms in a patient presenting with early stage Parkinson's Disease symptoms comprising:
determining a level of expression of at least one gene selected from the group consisting of SKP1a, UBE2K, ALDH1A1, PSMC4, HSPA8 and LAMB2 in a biological sample from a patient,
from the level of expression of the at least one gene, determining whether the patient's disease or symptoms are predicted to progress rapidly or slowly, and
administering to the patient a therapeutic effective amount of a symptom reducing medication appropriate for the slow or rapid progression of the symptom,
thereby reducing Parkinson's disease symptoms in a patient with slow or rapid progression of the symptoms or disease.

19. The method of claim 18, wherein rapid progression of Parkinson's Disease symptoms is treated with levodopa.

20. The method of claim 18, wherein determining the rapid progression is associated with dyskinesia.

21. The method of claim 20, wherein the rapid progression of dyskinesia is treated with symptom reducing medication selected from the group consisting of: low doses of levodopa, extended release of levodopa, continuous release of levodopa, and amantadine.

22. The method of claim 18, wherein determining the rapid progression is associated with cognitive decline.

23. The method of claim 22, wherein the symptom reducing medication of cognitive decline comprises: an atypical antipsychotic, a cholinesterase inhibitor, or a glutamine inhibitor.

24. The method of claim 18, wherein slow progression of the disease is not treated with symptom reducing medication selected from the group consisting of: levodopa, or carbidopa-levodopa.

25. The method of claim 18, wherein slow progression of the disease is treated with symptom reducing medication comprising: dopamine agonists and/or MAO-B inhibitors.

26. The method of claim 18, further comprising surgical therapies comprising: deep brain stimulation or intentional formation of lesions.

27. The method of claim 18, wherein the symptoms are selected from the group consisting of: resting tremor, bradykinesia, cognitive decline, rigidity, asymmetric resting tremor and asymmetric bradykinesia.

28. The method of claim 18, wherein determining whether the patient's disease or symptoms are predicted to progress rapidly or slowly comprises correlating the level of gene expression with a Parkinson's Disease rating scale.

29. The method of claim 28, wherein the Parkinson's Disease rating scale is selected from the group consisting of: Hoehn and Yahr scale (H&Y), Modified Schwab and England Activities of Daily Living (Modified Schwab and England), and Unified Parkinson Disease Rating Scale (UPDRS).

30. The method of claim 29, wherein a lower Modified Schwab and England Scale and/or increased H&Y score correlates with decreased baseline expression of the gene selected from the group consisting of ALDH1A1, LAMB2, SKP1a, and UBE2K prognoses the patient as having rapid progression of the symptoms or disease.

31. The method of claim 30, wherein the correlation is determined by a CL-Prediction algorithm comprising: (1.5479*ΔΔCT ALDH1a)+(−0.91861*ΔΔCT LAMB2)+(−0.21651*ΔΔCT UBE2K)+(1.15002*ΔΔCT SKP1a)+(0.11518*Age)−12.4435=predictive probability (PP) value, indicating rapid or slow prognosis of the disease.

32. The method of claim 31, wherein a positive PP value indicates a rapid progression of the disease and negative PP value indicates slow progression of the disease.

33. The method of claim 22, wherein the degree of cognitive decline is measured by at least one cognitive rating scale selected from the group consisting of: Montreal Cognitive Assessment (MoCA), Mini Mental State Examination (MMSE), Hopkins Verbal Learning Test (HVLT) and University of Pennsylvania Smell Identification Test (UPSIT).

34. The method of claim 33, wherein the cognitive decline correlates with increased expression of the gene selected from the group consisting of HSPA8 and SKP1a.

35. The method of claim 34, wherein the correlation is determined by the algorithm comprising: (−2.39361*ΔΔCT HSPA8)+(−1.27186*ΔΔCT SKP1a)+(0.15828*Age)+(−0.11884*UPSIT)+1.54744=PP value, indicating rapid or slow cognitive decline.

36. The method of claim 34, wherein the correlation is determined by the algorithm comprising: (−2.36482*ΔΔCT HSPA8)+(−1.39981*ΔΔCT SKP1a)+(−0.18071*Age)−1.8556=PP value, indicating rapid or slow cognitive decline
